(19) United States
(12) Patent Application Publication
(10) Pub. No.: US 2015/0045727 A1
(43) Pub. Date: Feb. 12, 2015

(54) CAPACITIVE NFC-BASED FILL LEVEL SENSOR FOR INSULIN PENS

(71) Applicants: AIT AUSTRIAN INSTITUTE OF TECHNOLOGY GMBH, WIEN (AT); SEIBERSDORF LABOR GMBH, SEIBERSDORF (AT)

(72) Inventors: Manfred Bammer, Wien (AT); Gernot Schmid, Bromberg (AT)

(21) Appl. No.: 14/386,960

(22) PCT Filed: Mar. 8, 2013
(86) PCT No.: PCT/AT2013/050060

§ 371 Date: Sep. 22, 2014

(30) Foreign Application Priority Data
Mar. 22, 2012 (AT) A 358/2012

(51) Int. Cl. A61M 5/168; G01F 23/26; A61M 5/145

(52) U.S. Cl. CPC A61M 5/1684; A61M 5/1452; G01F 23/263; A61M 2205/3317; A61M 2205/3576
USPC 604/67; 73/304 C

(57) ABSTRACT

A device determines a capacitance between two electrodes. The device contains a measurement circuit disposed downstream of the electrodes to determine the capacitance between the two electrodes, a communication unit disposed downstream of the measurement circuit, and a first antenna, connected to the communication unit, and having a coil configuration and a winding. The communication unit is configured to transmit its readings to an external data communication unit. The device further contains a second antenna having a coil configuration and a winding connected to the measurement circuit. The connections of the second antenna are connected to the electrodes, such that, when the antenna is excited with an electromagnetic alternating field, there is an alternating current on the electrodes. The measurement circuit performs a measurement of the alternating current on or flowing through the electrodes and the output of the measurement circuit is supplied to the communication unit.

The invention concerns a device for determining the capacity between two electrodes according to the generic terms of independent claim 1, as well as a device for determining capacity according to the generic terms of independent claim 12.

Numerous devices for administering fluids are known from the prior art. Such devices are primarily used for administering medications to persons or animals. The administration of insulin to diabetics, or applications in which the dosage of medications, hormones, biologicals, etc. is an important factor, in particular, is a preferred application for devices according to the invention.

Administration devices known as ‘insulin pens’ for administering insulin in the form of a liquid to diabetics are known from the prior art. With such devices, insulin can be simply and safely administered in the form of a liquid in the required amount to an individual patient; patients can control the administration themselves. Generally, the administration devices each have an ampoule with the respective gaseous or liquid medication, here insulin. These ampoules are often described as cartridges. The cartridges are inserted into the administration device, whereby an injection assembly obtains the medication from the respective cartridge and delivers it to the patient.

The injection assembly also has a dosing assembly that delivers a certain amount of the respective medication to the patient. Here, there is the problem that the dosing does not always work properly if the necessary amount of the medication is not present in the respective cartridge or ampoule. Although the most products allow the respective level of the cartridge or ampoule to be read via an inspection window, this only allows for an approximate determination of the amount of insulin or fluid in the cartridge or ampoule. In most cases, readings taken from the inspection window only allow for a very gross reading. It is difficult or impossible for patients with visual impairments to reliably determine the remaining amount of insulin or other fluids. Another problem arising in many applications is remembering the last fill level or the amount of the last injection. This is a problem not only for forgetful people, and can result in under- or overdose.

In order to remedy the first problem, the principle of capacitive fill level measurement is known from the prior art. To this end, at least two electrodes are attached either to the ampoule or cartridge itself or the inside of the lower pen shaft in the area of the ampoule. The attachment of the electrodes may be carried out by evaporation deposition or adhesion; in the case of adhesion or evaporation deposition on the inside of the pen shaft, the ampoule inserted into the pen should fit closely with the inside of the pen shaft. Due to the significant differences in the dielectric properties of the fluid, in particular insulin, and the dielectric properties of the non-metallic materials surrounding the fluid, the capacity Cm measured by both electrodes depends on the fill level in the ampoule. The ampoule or cartridge is referred to in relation to this invention as a fluid container.

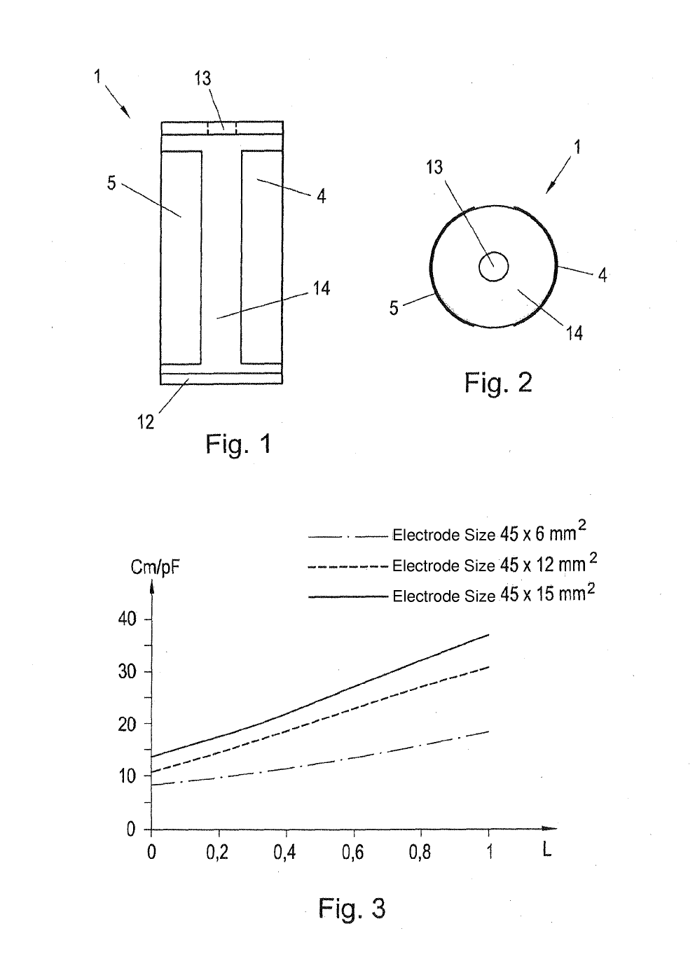

FIGS. 1-3 shows the underlying principle of determining the remaining amount of fluid L in a fluid container 1 in greater detail. FIG. 1 shows a side view of a fluid container 1 in the form of an ampoule, on the outer wall of which metal electrodes 4,5 are adhered or evaporation deposited. FIG. 2 shows a top view the fluid container 1. The metal electrodes 4, 5 are on circumferentially separate sections of the outer sheath of the cylindrical container 1 and do not come in contact with one another. FIG. 3 shows the relationship between the capacity measurable between the metal electrodes depending on the fill level of the fluid container 1 at different fill levels and different electrode 4, 5 sizes. By measuring the capacity between the two electrodes 4, 5 of the fluid container 1, the fill level L of the respective fluid 14, e.g., medication, in the fluid container 1 can be easily inferred to the extent that the permittivity of the fluid 14 differs sufficiently from the permittivity of air or the other fluid entering the container 1 instead of the fluid 14. In this case, the ratio of the two permittivities is approximately 1:80.

The problem with the method for capacitive fill level determination shown in FIGS. 1-3 is that this embodiment requires an AC voltage source to determine capacity, which causes problems in particular with components that are meant to be operated completely passively in conjunction with an NFC-capable mobile phone or another data communication device having an NFC interface. In particular, it requires considerable effort to integrate a battery to generate AC current to determine capacity.

Thus, the objective of the invention is to provide a device allowing for measuring the capacity of a capacitor in a passive component that is in particular NFC-compatible without requiring a separate power supply.

The invention achieves this in a device of the aforementioned type by means of the distinguishing characteristics of claim 1. In a device for the determination of capacity between two electrodes, the following components are provided:
a measurement circuit downstream of the electrodes to determine the capacity between the two electrodes,
a communication unit downstream of the measurement circuit, and
a first antenna, connected to the communication unit, having a coil configuration and at least one winding, whereby the communication unit is designed to transmit the readings it receives to an external data communication unit;
a second antenna having a coil configuration and at least one winding that is connected to the measurement circuit, whereby the connections of the second antenna are directly or indirectly connected to the electrodes, such that, when the antenna is excited with an electromagnetic alternating field, there is alternating current on the electrodes,
whereby the measurement circuit is configured for direct or indirect measurement of the alternating current on or flowing through the electrodes, and
whereby the output of the measurement circuit is supplied directly or indirectly to the communication unit.

In a method according to the invention, the essential advantage is that measurement of the capacity is possible without a separate AC generator and without a battery, and that the device according to the invention operates entirely passively. This also has the advantage that there is practically no measurable heating of the fluid in the ampoule, which can be critical for many temperature-sensitive injection solutions.

A particularly simple measurement of capacity provides respectively for one of the connections of the second antenna to be connected with one of the two electrodes, for the measurement circuit to be configured to determine the amplitude of the voltage between the two electrodes, and for it to keep a reading corresponding to this amplitude available at its output.

To determine measured values and capacities independently of the respective position and alignment of the communication device, it is advantageously provided for a third antenna having a coil configuration and at least one winding that surrounds the same surface area, and in particular has the same number of windings as the second antenna, a reference capacitor with a specified capacity, whereby the connections of the third antenna are directly or indirectly connected to the electrodes of the reference capacitor, such that, when the third antenna is excited with an electromagnetic alternating field, there is alternating current on the electrodes of the reference capacitor, a second measurement circuit for direct or indirect measurement of the alternating current on or flowing through the reference capacitor and a capacity or measurement determination unit upstream of the communication unit that determines the ratio of the readings obtained from the measurement circuits, in particular subjecting them to a calibration function and/or converting them to a reading derived from the capacity and outputting this at its output, whereby the communication unit is designed to transmit this ratio as a respective reading for the amount of fluid remaining in the fluid container.

A particularly simple measurement of capacity provides respectively for one of the connections of the third antenna to be connected with one of the two electrodes of the reference capacitor, and for a second measurement circuit downstream of the reference capacitor to be configured to determine the amplitude of the voltage between the two electrodes of the reference capacitor, which is connected to the output of the second measurement circuit.

A simply formed, robust embodiment of the invention allowing for reliable determinations provides for the second antenna and the third antenna, and, if applicable, the antenna, to surround the same surface area.

An advantageous variant of the invention can be used to determine the fluid content in a fluid container. Here, a fluid container is provided on the, in particular inner or outer, surface of which the two electrodes are arranged opposite and out of contact with one another, whereby preferably the capacity between the two electrodes depends on the amount of fluid in the fluid container and whereby the reading provided by the measurement circuit corresponds to the amount of fluid in the fluid container.

To administer the fluid to a living organism, it may be provided for the fluid container to be fluidically connected with an administration device, preferably designed for injection of this fluid into a living organism.

For proper dosing of the fluid to be administered, it may be provided for the administration device to be controlled by a control unit to which the fill level reading of the fluid container is provided, whereby the control unit activates the administration device until the fill level of the fluid container has been reduced by a specified amount.

Additionally, the invention concerns a configuration having a device according to the invention and a data communication unit, whereby the data communication unit is designed to deliver electromagnetic waves to the first antenna, the second antenna, and any third antenna. Advantageously, the data communication unit consists of a mobile telephone. With this configuration, the capacity can be determined advantageously and without a power source arranged in the device.

For advantageous recording and processing of the measured data, it may be provided for the data communication unit to have a receiver unit to receive the readings provided by the device, as well as a storage medium for storing these readings.

To control the delivery of fluids, it may be provided for the data communication unit to have an additional control unit, which receives the reading of the fill level of the fluid container at specified intervals, determines the difference between the fill level and a fill level stored in its storage medium, and transmits a signal if this difference exceeds a specified threshold.

Additionally, the invention concerns a method for determining the capacity between two electrodes with a first antenna for data communication and a second antenna having a coil configuration and at least one winding that is directly or indirectly connected to the two electrodes, such that, when the antenna is excited with an electromagnetic alternating field, there is alternating current on the electrodes. The invention provides for the second antenna to be excited by a data communication device with an electromagnetic alternating field, thus applying AC current to the electrodes, for the capacity between the two electrodes to be determined using the AC current, and for the capacity or a value derived therefrom to be transmitted to the data communication device via the first antenna.

In the method according to the invention, the advantage is that measurement of the capacity is possible without a separate AC generator and without a battery, and that the device according to the invention operates entirely passively. This also has the advantage that there is practically no measurable heating of the fluid in the ampoule, which can be critical for many temperature-sensitive injection solutions.

To determine measured values and capacities independently of the respective position and alignment of the communication device, it is advantageously to provide for a third antenna, in particular surrounding the same surface area as the second antenna, as well as a reference capacitor with a specified capacity, whereby the connections of the third antenna are directly or indirectly connected to the electrodes of the reference capacitor, such that, when the third antenna is excited with an electromagnetic alternating field, AC current is applied to the electrodes of the reference capacitor, whereby the third antenna is excited together with the second antenna by the data communication device with an electromagnetic alternating field, thus applying AC current to the electrodes, the capacity of the reference capacitor is determined using the AC current, and the ratio between the capacity of the two electrodes and the reference capacitor is transmitted to the data communication device via the first antenna.

An advantageous variant of the invention can be used to determine the fluid content in a fluid container. Here, a fluid container is provided on the, in particular inner or outer, surface of which the two electrodes are arranged opposite and out of contact with one another, characterised in that the capacity between the two electrodes or the ratio between the capacity between the two electrodes and the reference capacitor is used as a metric for the fill level, and is converted to a fill level in particular by means of a calibration table.

In order to query and monitor the individual dosages, it may be provided that the fill level of the fluid container is transmitted to the data communication unit, and that the fill level transmitted is stored in the data communication unit or another data communication device connected to it, and can be retrieved at a later time.

For proper dosing of the fluid to be administered, it may be provided for the fill level of the fluid container to be transmitted to the data communication unit, for the fluid container to be subsequently emptied, whereby the fill level of the fluid container is determined in real time, in particular at specified intervals, and transmitted to the data communication unit, for the difference between the fill level before the beginning of the emptying process and the last fill level transmitted to be determined, and for the data communication unit

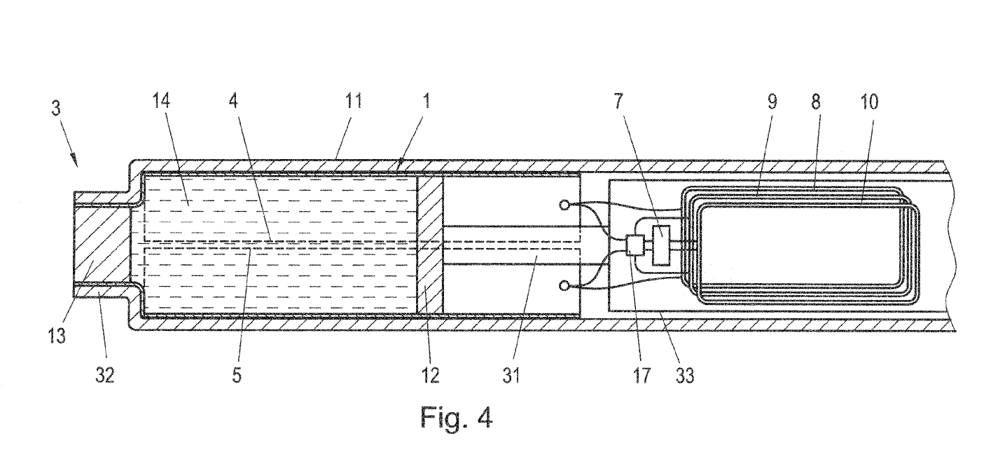

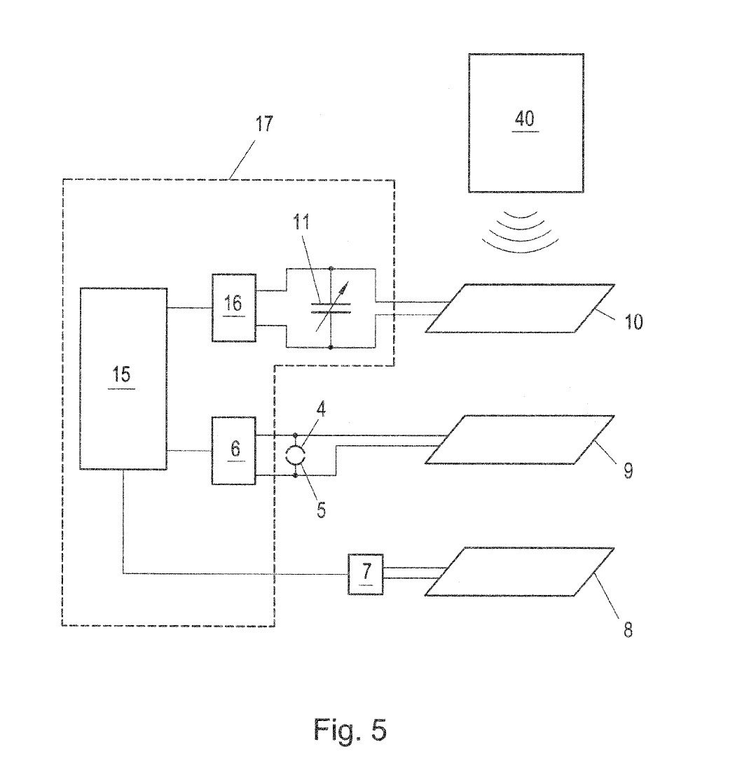

FIG. 1 shows a first representation of a side view of a fluid container. FIG. 2 shows a top view of the fluid container of FIG. 1. FIG. 3 shows the relationship between the capacity between the two electrodes and the fill level of the fluid container at different electrode sizes. An exemplary embodiment of the invention will be discussed in greater detail by reference to the following drawings. FIG. 4 shows a section of a device according to the invention in the form of an insulin pen. FIG. 5 is a schematic view of the electrical wiring of the individual components needed to determine capacity.

FIG. 4 shows an advantageous embodiment of the invention for determining capacity in greater detail. The device shown in FIG. 4 comprises a cylindrical housing 11, in which a fluid container 1 (FIGS. 1, 2), also cylindrical, is inserted. The fluid container 1 may be removed from the housing 11 and replaced with a fluid container 1 of the same type. On the outer sheath of the fluid container 1, electrodes 4, 5 are arranged that run along the fluid container 1. In this preferred exemplary embodiment, the two electrodes 4, 5 are spaced apart circumferentially (FIG. 2) and extend over the entire length of the fluid container 1. The fluid container 1 further comprises an end wall 12, arranged on a front side of the cylindrical fluid container 1. A recess 13 is arranged on the front surface of the cylindrical fluid container 1 opposite the end wall 12. Inside the fluid container 1, there is a fluid 14 to be administered to a person. This fluid 14 may escape and/or be removed from the fluid container 1 via the recess 13. By moving the end wall 12 relative to the sheath of the cylindrical fluid container 1, fluid 14 is pressed out of the fluid container 1, and the fluid-filled volume 14 of the fluid container 1 is reduced.

Additionally, the device of FIG. 4 has an administration device 3 or delivery unit by which fluid 14 can be administered from the fluid container 1 to a patient. An injection needle, in particular, is used as an administration device 3. In this preferred embodiment of the invention, the administration device 3 comprises a plunger 31 that pushes an end wall 12 of the fluid container 1 that is normal to the axial direction of the cylindrical fluid container 1 into the fluid container 1, thus moving fluid 14 on the end opposite this end wall 12 through a recess 13 to an injection area 32 of the administration device 3. The injection area 32 and the fluid container 1 are in direct fluidic connection. The administration device 3 further comprises a drive 33 for the plunger, which pushes the plunger 31 against the end wall 12 of the fluid container 1, thus administering the fluid 14 contained in the fluid container 1 to the respective patient.

The area between the end wall 12 and the recess 13 opposite the end wall 12 is completely filled with fluid 14; the remaining area of the fluid container 1 is empty, and filled with air in this case. By emptying the fluid container 1, the fluid 14, having a permittivity between 40 e₀ and 80 e₀, is successively replaced by water, which has a permittivity of approximately e₀. By reducing the permittivity of the intermediate space between the electrodes 4, 5, the capacity between the electrodes 4, 5 on the sheath of the fluid container 1 is also reduced. The reduction in capacity between the two electrodes 4, 5 caused by the emptying is shown in greater detail in FIG. 3.

The embodiment shown allows a fill level to be determined by capacitive measurement. However, the invention is not generally limited to the fill level measurement shown; rather, it can be used generally to measure any capacity or value, a change in which causes a change in capacity. Thus, it is not necessary for the invention to be used to determine a fill level. Instead, the invention allows for any capacity determination. Below, a capacity determination not requiring an additional AC power source or battery is described.

In this exemplary embodiment of the invention, the capacity between the two electrodes 4, 5 is measured by the circuit shown in FIG. 5. The circuit comprises an antenna 8 connected with a communication unit 7. The antenna 8 is a coil antenna, as used, e.g., in NFC applications. On the one hand, the antenna 8 serves to allow communication between the communication unit 7 and an external data communication device 40, e.g., a mobile telephone, and on the other hand, it allows the transmission of the energy required for measurements and communication from the data communication device 40 to the capacity determination unit according to the invention.

In a specific embodiment, the communication unit 7 may have a small buffer for intermediate storage of the amount of electrical energy required to operate the communication unit 7 during measurement and communication with the data communication device 40. However, the buffer need not be large enough for its energy content to be sufficient to generate an AC signal to determine the capacity between the two electrodes 4, 5.

Generally, the capacity between the two electrodes and the capacity of the reference capacitor 11 may be measured directly; the specific energy required for the measurement may be provided directly by the data communication device.

The preferred embodiment of the invention shown in this example has a second antenna 9 and a third antenna 10. The two connections of the second antenna are connected to the two electrodes 4, 5, There is a voltage between the two electrodes 4, 5, the amplitude of which depends from the capacity of the respective capacitor. The connections of the third antenna 10 are each connected to the electrodes of the reference capacitor 11.

In this case, the second antenna 9 and the third antenna 10 have the same number of windings. However, this is not required. Alternatively, for example, the selection of different numbers of windings can be used to create an intentional calibration offset, which simplifies the selection of the reference capacity as needed.

The electrical field energy contributed by the external data communication device 40 and the frequency of the field generated by the external data communication device 40 vary depending on the type of the external data communication device 40. In order to avoid different readings caused by the type of the external data communication device 40, the third antenna 10 is provided, and surrounds the same surface area as the second antenna 9. The two connections of the third antenna 10 are connected to the two electrodes of a reference capacitor 11. By comparing the voltage on the two electrodes 4, 5 with the voltage on the reference capacitor 11, a capacity reading independent of the respective external data communication device 40 can be obtained.

The second and third antennae 9, 10 surround the same surface area. The fanned view in FIGS. 4 and 5 is merely for the purposes of simplicity and ease of reference. Because the second and third antennae 9, 10 surround the same surface area, the respective relative position between the external data communication device 40 and the antennae 9, 10 has no influence on the ratio between the voltage on the output of the reference capacitor 11 and the voltage between the two electrodes 4, 5.

An essential advantage of the invention is that, to determine the capacity between the two electrodes 4, 5, and, if applicable, the capacity of the reference capacitor 11, no additional voltage generator or additional battery is required; the energy required to determine the respective capacity can be obtained directly from the magnetic field generated by the data communication device 40.

The preferred embodiment of the invention shown in this example has two measurement circuits 6, 16 that are connected to the communication unit 7 and transmit all values measured by them to the communication unit 7. In this case, the two measurement circuits 6, 16 each have a rectifier, a smoothing circuit downstream of the rectifier, and an ADC circuit downstream of the smoothing circuit. The result of the respective ADC circuit is provided to the communication unit 7.

A capacity and measurement determination unit 15 is provided that respectively determines the ratio between the voltage at the output of the reference capacitor 11 and the voltage between the two electrodes 4, 5 and keeps this result available at its output and transmits it to the communication unit 7. A conversion of this ratio into a capacity or a value derived therefrom may be performed, whereby the respective ratio is respectively compared with reference ratios determined in advance at specified capacity values. Thus, for example, the capacity value Cm obtained can be converted according to the diagram of FIG. 3 into the respective fill level L of the fluid 14 in the fluid container. The communication unit 7 transmits this ratio or the respective converted value on request to the external data communication device 40.

In this exemplary embodiment, the capacity and measurement determination unit 15, the measurement circuits 6, 16, and the reference capacitor 11 are arranged on a shared chip 17. In an alternative variant of the invention, the communication unit 7 may also be arranged on the chip 17.

In this exemplary embodiment of the invention, the administration device 3 is controlled by a control unit (not shown), to which the fill level reading of the fluid container 1 at the output of the comparison unit 15 is provided. The control unit activates the administration device 3 until the fill level of the fluid container 1 has decreased by a specified amount. The process may also be stopped if the plunger has reached a stop. In this case, an error message may be transmitted showing that the respective fluid container 1 is empty and that the required amount of fluid was not administered.

In order to determine what amount of fluid was administered at once, the fill level L is determined before and after administration as described above. Thus, a first fill level L1 before administration and a second fill level L2 after administration are available. If the difference DL between the first and second fill level is obtained, the result is the amount of fluid administered.

When administering a fluid, to dose the respective fluid, the first fill level L1 before administration can be saved and the second fill level can be determined in real time. If the difference DL between the first and second fill level reaches a threshold, administration is discontinued. To this end, the respective administration device 3 is deactivated; e.g., the drive 33 for the plunger 31 is deactivated and the plunger 31 is stopped. No more fluid 14 is administered or delivered.

1-16. (canceled)

17. A device for determining a capacitance between two electrodes, the device comprising:
a measurement circuit disposed downstream of the electrodes to determine the capacitance between the two electrodes;
a communication unit disposed downstream of said measurement circuit;
a first antenna connected to said communication unit and having a coil configuration and at least one winding, wherein said communication unit transmitting readings received by said communication unit to an external data communication unit;
a second antenna having a coil configuration and at least one winding connected to said measurement circuit, said second antenna having connections being directly or indirectly connected to the electrodes, such that, when said second antenna is excited with an electromagnetic alternating field, there is alternating current on the electrodes; and
said measurement circuit configured for direct or indirect measurement of the alternating current on or flowing through the electrodes, and an output of said measurement circuit is supplied directly or indirectly to said communication unit.

18. The device according to claim 17, wherein:
one of said connections of said second antenna is respectively connected to one of the two electrodes; and
said measurement circuit has an output, said measurement device determines an amplitude of a voltage between the two electrodes and keeps a value corresponding to the amplitude available at said output.

19. The device according to claim 17, further comprising:
a third antenna having a coil structure and at least one winding, surrounding a same surface area as said second antenna, and having a same number of windings as said second antenna;
a reference capacitor having a specified capacitance and electrodes, whereby said third antenna having connections directly or indirectly connected to said electrodes of said reference capacitor, such that, when said third antenna is excited with the electromagnetic alternating field, there is the alternating current on said electrodes of said reference capacitor;
a second measurement circuit for direct or indirect measurement of the alternating current on or flowing through said reference capacitor; and
a capacitance and/or measurement determining unit having an output and disposed upstream of said communication unit for determining a ratio of the readings obtained by said measurement circuits and subjecting the readings to a calibration function and/or converting the readings to a reading derived from the capacitance and outputting the reading at said output, wherein said communication unit transmits the ratio as a respective reading for an amount of fluid remaining in a fluid container.

20. The device according to claim 19, wherein one of said connections of said third antenna is respectively connected with one of said two electrodes of said reference capacitor, and said second measurement circuit downstream of said reference capacitor determines an amplitude of a voltage between said connections of said reference capacitor, whereby said communication unit has another output that is connected to said output of said second measurement circuit.

21. The device according to claim 19, wherein said second antenna and said third antenna surround a same surface area.

22. The device according to claim 17, further comprising a fluid container in which the two electrodes are disposed opposite and out of contact with one another, whereby the capacitance between the two electrodes depends on an amount of fluid in said fluid container, and whereby a reading provided by said measurement circuit corresponds to an amount of fluid in said fluid container.

23. The device according to claim 22, further comprising an administration device, wherein said fluid container is fluidically connected with said administration device configured for injection of the fluid into a living organism.

24. The device according to claim 23, further comprising a control unit, said administration device is controlled by said control unit to which a fill level reading of said fluid container is provided, whereby said control unit activates said administration device until the fill level of said fluid container has been reduced by a specified amount.

25. The device according to claim 19, wherein said first antenna, said second antenna and said third antenna surround a same surface area.

26. The device according to claim 22, wherein said fluid container has in an inner surface and an outer surface and the two electrodes are disposed on at least one of said inner surface and said outer surface.

27. A configuration, comprising:
a device for determining a capacitance between two electrodes, said device containing:
a measurement circuit disposed downstream of the electrodes to determine the capacitance between the two electrodes;
a communication unit disposed downstream of said measurement circuit;
a first antenna connected to said communication unit and having a coil configuration and at least one winding, wherein said communication unit transmitting readings received by said communication unit to an external data communication unit;
a second antenna having a coil configuration and at least one winding connected to said measurement circuit, said second antenna having connections being directly or indirectly connected to the electrodes, such that, when said second antenna is excited with an electromagnetic alternating field, there is an alternating current on the electrodes;
a third antenna;
said measurement circuit configured for direct or indirect measurement of the alternating current on or flowing through the electrodes, and an output of said measurement circuit is supplied directly or indirectly to said communication unit; and
a data communication unit configured to deliver electromagnetic waves to said first antenna, said second antenna, and said third antenna.

28. The configuration according to claim 27, wherein said data communication unit has a receiver unit to receive the readings provided by said device, and a storage medium for storing the readings.

29. The configuration according to claim 28, wherein said data communication unit has an additional control unit, which receives a reading of a fill level of a fluid container at specified intervals, determines a difference between the fill level and a fill level stored in said storage medium, and transmits a signal if a difference exceeds a specified threshold.

30. A method for determining a capacitance between two electrodes, which comprises the steps of:
providing a first antenna for data communication;
providing a second antenna having a coil configuration and at least one winding that is directly or indirectly connected to the two electrodes, such that, when the second antenna is excited with an electromagnetic alternating field, there is an alternating current on the electrodes;
exciting the second antenna with the electromagnetic alternating field by a data communication device, thus applying AC current to the electrodes;
determining from the AC current the capacitance between the two electrodes; and
transmitting the capacitance or a value derived from the capacitance to the data communication device via the first antenna.

31. The method according to claim 30, which further comprises:
providing a third antenna surrounding a same surface area as said second antenna;
providing a reference capacitor having a specified capacitance, whereby connections of the third antenna are directly or indirectly connected to the electrodes of the reference capacitor, such that, when the third antenna is excited with the electromagnetic alternating field, there is the alternating current on the electrodes of the reference capacitor;
exciting the third antenna together with the second antenna with the electromagnetic alternating field by the data communication device, thus applying the AC current to the reference capacitor;
determining from the AC current a capacitance of the reference capacitor; and
transmitting a ratio of the capacitance between the two electrodes and the reference capacitor to the data communication device.

32. The method according to claim 30, which further comprises:
providing a fluid container, the two electrodes disposed on one of an inner surface or an outer surface the fluid container, the two electrodes being disposed opposite and out of contact with one another; and
using the capacitance between the two electrodes or a ratio of the capacitance between the two electrodes and the reference capacitor as a metric for a fill level, and the ratio is converted to the fill level by a calibration table.

33. The method according to claim 32, which further comprises transmitting the fill level of the fluid container to the data communication unit, and that the fill level transmitted is stored in the data communication unit or another data communication device connected to it, and can be retrieved at a later time.

34. The method according to claim 33, which further comprises:
transmitting the fill level of the fluid container to the data communication unit; and
emptying the fluid container, whereby the fill level of the fluid container is determined in real time, in specified intervals, and transmitted to the data communication unit, a difference between the fill level before a beginning of an emptying process and a last fill level transmitted is determined and the data communication unit transmits a signal when a difference exceeds a specified threshold and an emptying of the fluid container is interrupted after the signal is transmitted.
